(19) United States
(12) Patent Application Publication
(10) Pub. No.: US 2025/0261999 A1
(43) Pub. Date: Aug. 21, 2025

(54) SYSTEM FOR OPTICALLY MEASURING A SUBCUTANEOUS IMPLANTATION DEPTH OF AN IMPLANT

(71) Applicant: Synergia Medical, Mont-Saint-Guibert (BE)

(72) Inventors: Pascal Doguet, Mont-Saint-Guibert (BE); Jérôme Garnier, Mont-Saint-Guibert (BE); Arnaud Rueff, Mont-Saint-Guibert (BE)

(73) Assignee: Synergia Medical, Mont-Saint-Guibert (BE)

(21) Appl. No.: 18/858,446

(22) PCT Filed: Apr. 26, 2022
(86) PCT No.: PCT/EP2022/060993

§ 371 Date: Oct. 21, 2024

(51) Int. Cl. A61B 34/20; A61B 90/00; A61N 1/372; G16H 40/67

(52) U.S. Cl. CPC A61B 34/20; G16H 40/67; A61B 2034/2055; A61B 2090/062; A61N 1/372

(57) ABSTRACT

A system for optically measuring an implantation depth (d) of an active implantable medical device (AIMD) below a skin (3s) of a patient is provided. The AIMD (20) includes light sources for emitting light beams towards the outer environment along at least two non-parallel emission axes forming first and second axis angles to form a light projection at a level of the skin characterized by first and second maxima separated from one another by a maxima distance. An external optical depth measuring device (40) is provided that includes at least two photodetectors configured for detecting the maxima, and includes an intelligence configured for measuring the maxima distance between the maxima at the level of the skin of the patient, and determining from the maxima distance the implantation depth.

### TECHNICAL FIELD

The present invention concerns a system for determining an implantation depth of an active implantable medical device (AIMD) in a body of a patient. The implantation depth is measured with optical light beams emitted from the implanted AIMD and reaching a skin of the patient. Knowing the implantation depth is important to optimize the circuit of an external charger for charging a battery located in the AIMD, such as to approach, preferably reach resonance between an implanted secondary coil (=receiver) enclosed in the AIMD and an external primary coil (=emitter) in the external charger. Approaching, preferably reaching the resonance between the external charger and the AIMD is desired to reduce charging time of the battery and enhance the external charger autonomy.

### BACKGROUND OF THE INVENTION

Many types of active implantable medical devices (AIMD) exist and have various functions. Once implanted, they must cooperate with an external element to continue their function. In particular, for AIMD's consuming electrical energy and enclosing an implanted rechargeable battery, such as neurostimulators, the implanted battery must be replenished at regular intervals.

As shown in FIG. 1, transcutaneous (wireless) charging of an implanted battery (25) lodged in an implanted AIMD (20) can be carried out across the skin (3s) of the patient with an external charger (10) by inducing an alternative current in a secondary coil (21) of the implanted AIMD by means of a magnetic field (B) generated by an alternative current circulating in a primary coil (11) of the external charger (10). In the following, unless otherwise specified, the term “current” is used to define an alternative current.

The power transfer efficiency (P0/P1) defined as a ratio of the power (P0) received at the receiver (22) of the implanted AIMD, to the power (P1) consumed at the transmitter (12) of the external element (10) strongly depends on a number of parameters, including the alignment of the primary and secondary coils (11, 12), the tilting angle of deviation from parallelism between the two coils, and also the distance (h) separating the primary coil (11) from the secondary coil (22).

Positioning the external charger (10) to bring the primary coil (11) in perfect alignment with the secondary coil (21) can be achieved by means known in the art. For example, WO2017202455 describes a system for centring the primary coil (11) of an external charger (10) with respect to the secondary coil (21) of an implanted AIMD (20) by providing the implanted AIMD with a light source positioned such as to emit a light beam towards the skin of the patient, the light beam being coaxial to a secondary axis (Z2) centred on the secondary coil (21). The external charger comprises at least three photodetectors forming a polygon normal to and centred on the primary axis (Z1), which is itself centred on the primary coil (11). The primary and secondary coils (11, 21) are coaxial when an equal optical energy is received from the light beam by all the photodetectors of the external charger. Any difference in optical energy between two photodetectors is indicative of an offset between the primary and secondary axes (Z1, Z2).

In many applications, such as for vagus nerve stimulation (VNS) or for deep brain stimulation (DBS), wherein the AIMD is implanted, respectively, in the sub-clavicular area or between the skull and skin of the patient, the AIMD is generally substantially parallel to the skin and therefore so are the secondary coil (21) and the primary coil (11) of the AIMD (20) and external charger (10) (i.e., the tilting angle is substantially nil.

The power transfer efficiency (P0/P1) also depends on the distance (h) separating the primary coil (11) from the secondary coil (21). Each one of the transmitter circuit comprising the primary coil (11) and the receiver circuit comprising the secondary coil (21) includes one or more charging capacitors to yield capacitance values precisely tuned to enhance the resonance between the transmitter circuit and the receiver circuit. Resonance occurs when the natural frequency of a system is the same as the frequency of an oscillating force applied to the system. Its effect is to maximize the amplitude of the oscillating response of the system. The oscillating force is the electromagnetic force associated with the oscillating magnetic field (B) created by powering the primary coil (11) at a constant frequency. The natural frequency of the system is variable and depends on several parameters, including the distance (h) between the primary and secondary coils (11, 21). To enhance the resonance, the capacitance values of the external charger shall be adapted and tuned as a function of the actual distance (h). Since in a system composed of an external charger (10) laid on the skin of the patient over an implanted AIMD (20), the distance (h) is equal to, or univocally related to the value of the implantation depth of the AIMD, in the following, the terms “distance” (h) and “implantation depth” (h) are used interchangeably as synonyms.

FIG. 2 illustrates the power transfer efficiency (P0/P1) as a function of the implantation depth (h). The dashed line labelled “C2” characterizes the P0/P1 vs h plot for a prior art external charger comprising a single charging capacitor (C2) of fixed capacitance. It can be seen that the power transfer efficiency (P0/P1) varies strongly with the implantation depth (h). Taking arbitrarily P0/P1=25% (cf. horizontal straight line) as a minimum power transfer efficiency (P0/P1) below which the power transfer is considered as not sufficiently efficient, it can be seen that the external charger of the prior art labelled “C2” can only be used in a limited C2-implantation depth-range (hC2) comprised between hc2.0 and hc2.1.

The implantation depth (h) which an AIMD will be implanted at is not known in advance and the optimization of the capacitance values of the external charger cannot be carried out during production of the AIMD in plant. In general, the external charger and AIMD are produced with capacitance values which are reasonably optimized for a limited range of implantation depths (h) which is statistically representative of actual implantation depths. The problem remains to enhance resonance in all cases, including the cases wherein the implantation depth falls out of the limited range (hC2) of implantation depths (h).

A skilled person faced with the foregoing problem must first find a way to assess the implantation depth (h) of the AIMD and, second, optimize the capacitance value of the external charger (10) to the thus assessed implantation depth to enhance resonance of the system.

The present invention proposes an original, reliable and simple solution for solving this dual problem of assessing the actual implantation depth (h) and optimizing the capacitance value of the external charger to the actual implantation depth. These and other advantages of the present invention are presented in the next sections.

### SUMMARY OF THE INVENTION

The present invention is defined in the appended independent claims. Preferred embodiments are defined in the dependent claims. In particular, the present invention concerns a system for optically measuring an implantation depth (d) of an active implantable medical device (AIMD) below a skin of a patient implanted with the AIMD, the system comprising the AIMD and an external optical depth measuring device.

The AIMD (20) comprises an encapsulation unit suitable for being subcutaneously implanted with a reference plane (R) of the encapsulation unit being substantially parallel to the skin of the patient when implanted in the patient and comprising a housing comprising walls defining an inner volume sealed from an outer environment by the walls and defining a first main surface configured for facing the skin of the patient when implanted in the patient. One or more light sources are provided, which are configured for emitting one or more light beams from the main surface towards the outer environment to form a light projection pattern at a level of the skin of the patient, characterized by an intensity profile comprising at least first and second maxima separated from one another by a maxima distance (ds).

The external optical depth measuring device (40) comprises a controller and two or more photodetectors. The controller is configured for activating the one or more light sources of the AIMD. The two or more photodetectors are configured for detecting the at least first and second maxima of the light projection pattern emitted by the one or more light sources.

The gist of the invention is that the one or more light sources are configured for emitting one or more light beams along at least two emission axes (X1, X2) defined as the axes extending from one or more emission points of the one or more light sources to the corresponding at least first and second maxima of the light projection pattern at the level of the skin of the patient. The at least two emission axes (X1, X2) are non-parallel to one another, and form at least first and second axis angles (α1, α2) relative to a normal axis (n1; n2) perpendicular to the reference plane (R), each comprised between 0° and 88°, preferably between 10° and 60°, more preferably between 20° and 45°. The external optical depth measuring device comprises an intelligence configured for,
determining the respective positions of the first and second maxima,
measuring the maxima distance (ds) between the first and second maxima at the level of the skin of the patient, and
determining from the maxima distance (ds) the implantation depth (h) of the AIMD below the skin of the patient.

The implantation depth (h) can be determined with any one of the following expressions (1) or (1a),

$$h = \frac{ds}{tan α1 + tan α2} - h 0 , or \qquad ( 1 )$$

$$h = \frac{ds - d 0}{tan α1 + tan α2} \qquad ( 1 a )$$

wherein
h0 is a distance measured along a central normal axis (n0) perpendicular to the reference plane (R) between the intersection point of the first and second emission axes (X1, X2) and the main surface (20m), and
d0 is a distance separating the first from the second light sources (51, 52).

In a preferred embodiment, the first and second axis angles (α1, α2) are equal (i.e., (α1=α2). By introducing α1=α2 into Equations (1) or (1a), the implantation depth (h) can be determined with any one of the following expressions (3) or (3a),

$$h = \frac{ds}{2 tan α1} - h 0 \qquad ( 3 )$$

$$h = \frac{ds - d 0}{2 tan α1} \qquad ( 3 a )$$

In an alternative embodiment, the first axis angle (α1) is equal to zero (i.e., α1=0) and is different from the second axis angle (α2). Introducing α1=0 into Equations (1) or (1a), the implantation depth (h) can be determined with any one of the following expressions (4) or (4a),

$$h = \frac{ds}{tan α2} - h 0 \qquad ( 4 )$$

$$h = \frac{ds - d 0}{tan α2} \qquad ( 4 )$$

The one or more light sources can be formed either by,
at least first and second light sources separated from one another by a distance (d0) and each configured for emitting a light beam along the corresponding at least first and second emission axes (X1, X2), or
a single light source configured for emitting a light beam characterized by the at least first and second maxima.

In a preferred embodiment, the system comprises an external charger comprising or connectable to a source of power for feeding alternative current to a primary coil made of one or more wires coiled about a primary axis (Z1) and forming an external emitter of magnetic waves. In this embodiment, the AIMD comprises a secondary coil made of one or more wires coiled about a secondary axis (Z2) normal to the reference plane (R) and forming an internal receiver. With this configuration, when the electric charger is located adjacent to the AIMD with the primary axis (Z1) positioned coaxially to the secondary axis (Z2) and when the primary coil is fed with alternative current, a magnetic link is formed and an alternative current induced in the secondary coil is optimized, which serves to power the AIMD or to recharge a rechargeable battery enclosed in the inner volume (Vi). The external charger can comprise,
an electrical circuit configured for varying the properties of the alternative current fed to the primary coil,
a processor configured for retrieving the implantation depth (h) of the AIMD measured by the external optical depth measuring device and for controlling the alternative current fed to the primary coil to optimize a power transfer efficiency between the primary coil of the external charger and the secondary coil of the AIMD as a function of the implantation depth (h).

In a preferred embodiment, the external optical depth measuring device is included in the external charger and the intelligence is included in the processor.

In particular, the electrical circuit can comprise at least first and second charging capacitors (C1, C2) of given capacitances, which can be individually connected or disconnected to the electrical circuit. The processor can be configured for optimizing the power transfer efficiency between the primary coil of the external charger and the secondary coil of the AIMD as a function of the implantation depth (h) by selecting which of the first and/or second capacitance (C1, C2) to connect to the electrical circuit.

In a preferred embodiment, the electrical circuit comprises N charging capacitors (C1-CN) of same or different charging capacitors, which can be connected or disconnected individually or in groups of charging capacitors to the electrical circuit. The processor can be configured for determining which combination of one or several of the N charging capacitors (C1-CN) to connect to the electrical circuit, such as to optimize the power transfer efficiency as a function of the implantation depth (h). the number N of capacitors can be comprised between 3 and 8, preferably between 4 and 6.

The present invention also concerns a method for determining an implantation depth (h) separating an external optical measuring device from an active implantable medical device (=AIMD) implanted in a patient, comprising the following steps,
providing an AIMD as defined supra, whose main surface is separated from an outer environment by a distance to be determined, by a skin substantially parallel to the reference plane (R),
applying an external depth measuring device as defined supra onto the skin, vis-à-vis the AIMD,
activating with the controller the one or more light sources of the AIMD,
measuring the maxima distance (ds) and determining therefrom the value of the distance.

For optimizing a power transfer efficiency between a primary coil of an external charger and a secondary coil of the AIMD as a function of the implantation depth (h), the method mat further comprise,
providing an external charger as defined supra,
retrieving the thus determined distance, and
determining an optimal alternative current to be fed to the primary coil as a function of the distance, to optimize the power transfer efficiency (P0/P1) between the primary coil of the external charger and the secondary coil of the AIMD,
applying the external charger onto the skin vis-à-vis the AIMD and feeding the optimal alternative current to the primary coil.

Controlling the alternative current fed to the primary coil can comprise selecting one or more charging capacitors (C1-CN) to connect to the electrical circuit, such as to optimize the power transfer efficiency between the primary coil of the external charger and the secondary coil of the AIMD as a function of the distance thus determined.

### BRIEF DESCRIPTION OF THE FIGURES

For a fuller understanding of the nature of the present invention, reference is made to the following detailed description taken in conjunction with the accompanying drawings in which:

FIG. 1: shows the principle of inductive charging of a battery lodged in an implanted AIMD by means of an external charger.

FIG. 2: compares the power transfer efficiency (P0/P1) between a system of the prior art with a single charging capacitor (C2) of constant capacitance value (=dashed line) with a system of the present invention comprising four charging capacitors (C1-C4) in parallel, allowing the capacitance of the electrical circuit of the external charger to be optimized as a function of the implantation depth (h).

FIG. 3 (a): illustrates the geometric principle of implantation depth measurement with the system of the present invention, with two light maxima for any value of α1 and α2.

FIG. 3 (b): plots the implantation depth (h) as a function of the measured maxima distance (ds) according to the Equation (1a).

FIG. 3 (c): plots the (ds/h) ratio of the maxima distance (ds) to the implantation depth (h) as a function of the value of the second axis angle (α2) for α1=30°.

FIG. 4: illustrates the geometric principle of implantation depth measurement with the system of the present invention, with two light maxima for the special case of α1=α2.

FIG. 5: illustrates the geometric principle of implantation depth measurement with the system of the present invention, with two light maxima for the special case of α1=0°.

FIG. 6: illustrates the geometric principle of implantation depth measurement with the system of the present invention with an annular light beam.

FIG. 7 (a): illustrates an external optical depth measuring device according to the present invention laid against the skin of a patient facing an implanted AIMD.

FIG. 7 (b): illustrates an external optical depth measuring device according to the present invention integrated in an external charging unit laid against the skin of a patient facing an implanted AIMD.

FIG. 8 (a): shows a representation of a simplified electrical circuit of an external charger according to the prior art.

FIG. 8 (b): shows a representation of a simplified electrical circuit of an external charger according to the present invention.

FIG. 9 shows a flowchart illustrating the optimization of the external charger after having determined the implantation depth (h) of the AIMD.

### DETAILED DESCRIPTION OF THE INVENTION

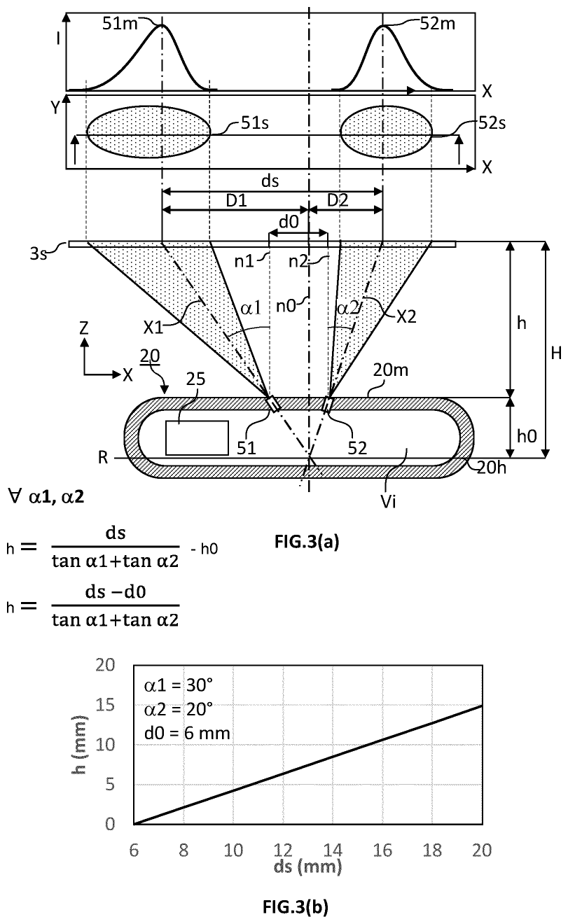

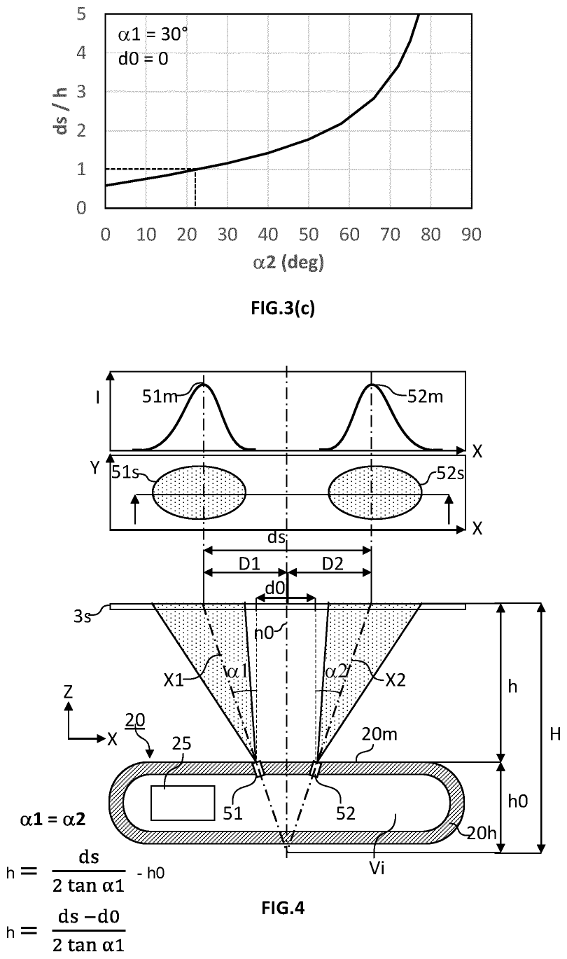

As illustrated in FIGS. 3 to 7, the system of the present invention is suitable for optically measuring an implantation depth (h) of an active implantable medical device (AIMD) (20) below a skin (3s) of a patient implanted with the AIMD. The system of the present invention comprises an AIMD and an external optical depth measuring device (40).

The AIMD (20) comprises an encapsulation unit (20e) and one or more light sources (51, 52). The encapsulation unit (20e) is suitable for being subcutaneously implanted with a reference plane (R) of the encapsulation unit being substantially parallel to the skin (3s) of the patient when implanted in the patient. It comprises a housing (20h) comprising walls defining an inner volume (Vi) sealed from an outer environment by the walls and defining a first main surface (20m) configured for facing the skin of the patient when implanted in the patient.

The one or more light sources (51, 52) are configured for emitting one or more light beams from the main surface (20m) towards the outer environment to form a light projection pattern (51s, 52s) at a level of the skin (3s) of the patient. The light projection pattern is characterized by an intensity profile comprising at least first and second maxima (51m, 52m) separated from one another by a maxima distance (ds). The AIMD can comprise first and second light sources (51, 52) as illustrated in FIGS. 3 to 5, which are separated from one another by a distance (d0) and each configured for emitting a light beam along the corresponding at least first and second emission axes (X1, X2). Alternatively, as shown in FIG. 6, the AIMD can comprise a single light source (51) configured for emitting a light beam characterized by the at least first and second maxima (51m, 52m). The single light source can emit two or more discrete light beams or a single light beam forming a ring as shown in FIG. 6

The external optical depth measuring device (40) comprises a controller configured for activating the one or more light sources of the AIMD, and two or more photodetectors (44) or an array of two or more photodetectors configured for detecting the at least first and second maxima (51m, 52m) of the light projection pattern emitted by the one or more light sources.

The one or more light sources are configured for emitting one or more light beams along at least two emission axes (X1, X2) defined as the axes extending from one or more emission points (51, 52) of the one or more light sources to the corresponding at least first and second maxima (51m, 52m) of the light projection pattern (51s, 52s) at the level of the skin (3s) of the patient. The at least two emission axes (X1, X2) are non-parallel to one another and form at least first and second axis angles (α1, α2) relative to a normal to the reference plane (R), each comprised independently from one another between 0° and 88°, preferably between 10° and 60°, more preferably between 20° and 45°.

The external optical depth measuring device comprises an intelligence (48) configured for,
determining the respective positions of the first and second maxima (51m, 52m),
measuring the maxima distance (ds) between the first and second maxima (51m, 52m) at the level of the skin (3s) of the patient, and
determining from the maxima distance (ds) the implantation depth (h) of the AIMD below the skin (3s) of the patient.

Once the implantation depth (h) has been assessed, an external charger (10) can be tuned to enhance resonance of the system between the primary and secondary coils (11, 21) of the external charger and AIMD, respectively. To this effect, the external charger (10) can comprise an electrical circuit (16) configured for varying the properties of the electric current fed to the primary coil (11). A processor (19) is configured for retrieving the implantation depth (h) of the AIMD (20) measured by the external optical depth measuring device (40) and for controlling the current fed to the primary coil (11) to optimize the power transfer efficiency between the primary coil (11) of the external electric charger (10) and the secondary coil (21) of the AIMD (20) as a function of the implantation depth (h). The current fed to the primary coil (11) can be controlled by varying the capacitance of the one or more charging capacitors (C1C4) of the transmitter circuit and/or the power supplied to the transmitter circuit.

### AIMD (20)

The active implantable medical device (20) (=AIMD) of the present invention comprises an encapsulation unit (20e) suitable for being subcutaneously implanted with a reference plane (R) of the encapsulation unit being substantially parallel to the skin (3s) of the patient when implanted in the patient. As illustrated in FIGS. 3 (a), 4 to 6, and 7 (a), the encapsulation unit comprises a housing (20h) comprising walls defining an inner volume (Vi) sealed from an outer environment by the walls and defining a first main surface (20m) configured for facing the skin of the patient when implanted in the patient.

According to the present invention, the encapsulation unit comprises one or more light sources (51, 52) configured for emitting one or more light beams from the main surface (20m) towards the outer environment to form a light projection pattern (51s, 52s) at a level of the skin (3s) of the patient, which is substantially parallel to the main surface (20m). As shown in FIGS. 3 to 6, the light projection pattern is characterized by an intensity profile comprising at least first and second maxima (51m, 52m) separated from one another by a maxima distance (ds). The one or more light sources are configured for emitting one or more light beams along at least two emission axes (X1, X2) defined as the axes extending from one or more emission points of the one or more light sources (51, 52) to the corresponding at least first and second maxima (51m, 52m) of the light projection pattern (51s, 52s) at the level of the skin (3s) of the patient. The at least two emission axes (X1, X2) are non-parallel to one another, and form at least first and second axis angles (α1, α2) relative to a normal to the reference plane (R), each comprised between 0° and 88°, preferably between 10° and 60°, more preferably between 20° and 45°.a

As shown in FIG. 6, a single light source can be used to emit two or more beams along the at least two emission axes (X1, X2). In FIG. 6, the single light source (51) is configured for emitting an infinite number of beams forming a continuous ring of light (cf. shaded areas of the doughnut shaped ring of light on top of FIG. 6. FIG. 6 shows a continuous ring which is circular but the light projection pattern can have other geometries such as a triangle or a rectangle, preferably a square, or any polygonal geometry. Alternatively, the single light source (51) can be configured to emit a finite number of at least two beams extending along at least corresponding first and second emission axes (X1, X2).

As shown in FIGS. 3 to 5 and 7, the encapsulation unit can comprise at least two distinct light sources (51, 52). The discussion of FIGS. 3 to 5 is based on two distinct light sources, but it is clear that the encapsulation unit can comprise more than two distinct light sources.

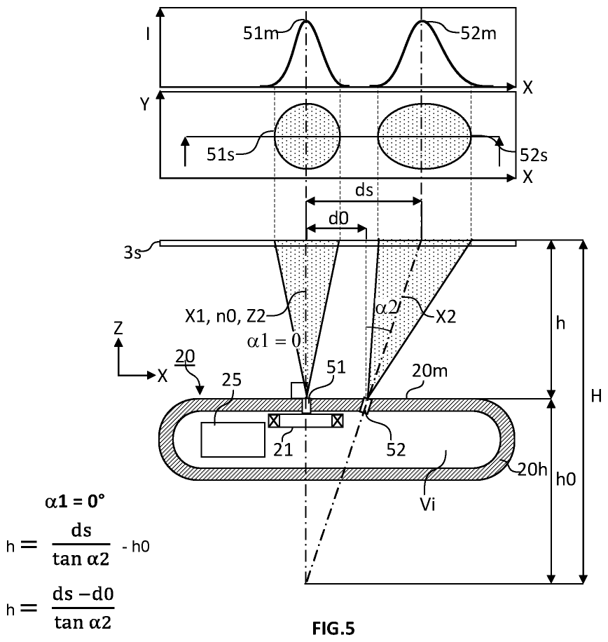

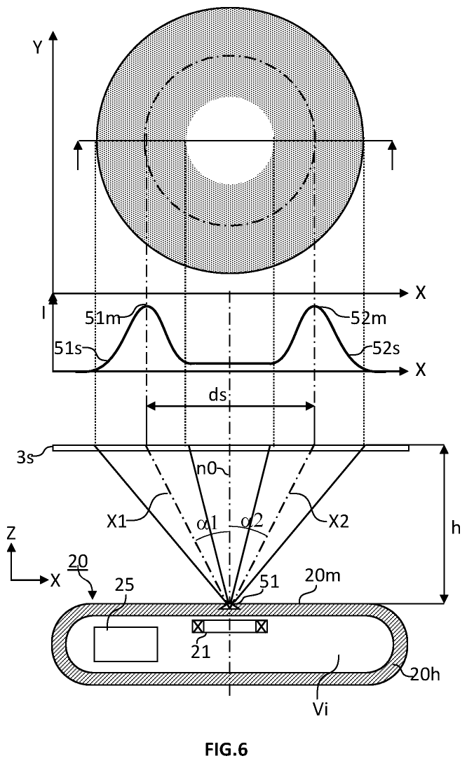

In FIG. 3 (a), the first and second axis angles (α1, α2) between the central normal axis (n0) perpendicular to the reference plane (R) and each of the first and second emission axes (X1, X2) can take any value comprised between 0° and 88°. FIG. 4 shows an embodiment wherein the first axis angle (α1) is equal to the second axis angle (α2), i.e. α1=α2. FIG. 5 shows an embodiment wherein the first axis angle (α1) is nil, i.e. α1=0. The special embodiments of FIGS. 4 and 5 simplify the equation for determining the implantation depth (h) as a function of the maxima distance (ds) as is discussed in detail in continuation.

The intensity of the one or more light sources (51, 52) must be sufficient to give clear first and second light projection patterns with clearly distinguishable and identifiable maxima, so as to yield an accurate value of the maxima distance (ds). Since the tissues separating the AIMD from the skin of the patient can be inhomogeneous, light is not only absorbed but also scattered as it travels through the tissues. The duration of the light beams can, however, be very short, since all that is required is to identify the positions of the first and second maxima (51m; 52m) by the photodetectors (44). As the light beam must have a sufficient intensity, saving battery power can be achieved by shortening the duration of the light beam.

The AIMD can be a neurostimulator for stimulating electrically or optically a tissue such as a nerve. For example, the AIMD can be a neurostimulator for vagus nerve stimulation (VNS) or deep brain stimulation (DBS). The encapsulation (20e) of such AIMD's is generally implanted at a distance from the tissue to be stimulated and comprises a tissue coupling unit separate from the encapsulation unit (20e) and configured for coupling an electrode unit or an optrode unit to the tissue. Energy pulses are generated from an implanted pulse generator (IPG) lodged in the inner volume (Vi) of the encapsulation unit (20e). The energy pulses are transferred to the tissue coupling unit via electric wires or optical fibres, such as described in WO2016131492.

As shown in FIGS. 5, 6, 7 (a) and 7 (b), the AIMD (20) comprises a secondary coil (21) made of one or more wires coiled about a secondary axis (Z2) normal to the reference plane (R) and forming part of an implanted receiver (22). It is conductively coupled to an implanted battery (25) and configured for charging the implanted battery when exposed to an alternative magnetic field (B) inducing an electric current through the secondary coil (21). The alternative magnetic field can be generated by an external charger (10) comprising a primary coil (11) made of one or more wires coiled about a primary axis (Z1) and forming an external emitter (12) of magnetic waves, as discussed in continuation.

### External Optical Depth Measuring Device (40)

The external optical depth measuring device (40) comprises a controller enclosed in a device housing (40h) and configured for activating the one or more light sources of the AIMD. In use, the external optical depth measuring device is laid onto the skin of the patient over the implantation location of the AIMD. In that position, the controller can communicate with the implanted AIMD and control the activation of the one or more light sources.

The external optical depth measuring device (40) comprises two or more photodetectors (44) or an array of two or more photodetectors configured for detecting the at least first and second maxima (51m, 52m) of the light projection pattern on the skin, emitted by the one or more light sources. The two or more photodetectors are preferably distributed on a single plane. In use, i.e., when the external optical depth measuring device (40) lies on the skin of the patient, over the AIMD, the single plane is preferably substantially parallel to the reference plane (R) of the AIMD. In case it is suspected or known that the AIMD is implanted such that the reference plane (R) thereof is not parallel to the skin, the tilting angle of the reference plane should be determined to correct the errors in implantation depth (h) determination due to the lack of parallelism of the single plane of the external optical depth measuring device (40) and the reference plane (R) of the AIMD (20). The external optical depth measuring device needs not be perfectly aligned with the AIMD, as long as the photodetectors (44) can detect at least first and second light (51m, 52m) maxima.

The external optical depth measuring device (40) also comprises an intelligence (48) configured for,
determining the respective positions of the first and second maxima (51m, 52m),
measuring the maxima distance (ds) between the first and second maxima (51m, 52m) at the level of the skin (3s) of the patient, and
determining from the maxima distance (ds) the implantation depth (h) of the AIMD below the skin (3s) of the patient, in ways as described in continuation.

The thus determined maxima distance (ds) is important to tune the external charger (10) for inductively charging the implanted battery of the implanted AIMD as is explained in continuation.

### Determination of the Implantation Depth (h)

The implantation depth (h) can be determined trigonometrically from the maxima distance (ds) separating two intensity maxima (51m, 52m) of the light projection pattern (51s, 52s) at the level of the skin (3s) of the light beams extending along the first and second emission axes (X1, X2). FIG. 3 (a) illustrates a general case, wherein two separate light sources emit light beams along the first and second emission axes (X1, X2) forming axis angles (α1, α2), which can take any value comprised between 0 and 88°.

The maxima distance (ds) is equal to the sum of D1 and D2 (i.e., ds=D1+D2). From FIG. 3 (a) it can easily be assessed that D2=D1 (tan α2/tan α1). It follows that D1=ds/(1+ (tan α2/tan α1)). Introducing this expression for D1 into the Equation, H=h+h0=D1/tan α1 and rearranging yields Equation (1) which defines the implantation depth (h) as a function of the measured maxima distance (ds),

$$h = \frac{ds}{tan α1 + tan α2} - h 0 \qquad ( 1 )$$

The parameters, α1, α2, and h0 are known from the AIMD's manufacturer. If the value of h0 is not readily available, it can easily be determined as a function of the distance (d0) separating the first and second emission points (51, 52) in a similar way as Eq. (1) yielding,

$$h 0 = \frac{d 0}{tan α1 + tan α2} \qquad ( 2 )$$

Introducing Eq. (2) into Eq. (1) yields Eq. (1a) expressing h as a function of ds, do, and α1, α2,

$$h = \frac{ds - d 0}{tan α1 + tan α2} \qquad ( 1 a )$$

Equations (1) and (1a) are valid for all values of α1 and α2 comprised between 0° and 88° independently of one another. The height (h0) is defined as the distance measured along a central normal axis (n0) perpendicular to the reference plane (R) between the intersection point of the first and second emission axes (X1, X2) and the main surface (20m). In case a single light source (51) emits beams along at least the first and second emission axes (X1, X2), the value of both h0 and do is zero (i.e., d0=h0=0), as shown in FIG. 6. Equations (1) and (1a) then simplify to Equation (1b),

$$h = \frac{ds}{tan α1 + tan α2} \qquad ( 1 b )$$

Equations (1) and (1a) define a linear relationship between the measured maxima distance (ds) and the implantation depth (h). FIG. 3 (b) illustrates an Example of curve relating the implantation depth (h) to the maxima distance (ds), with α1=30°, α2=20°, and d0=6 mm.

The axis angles (α1, α2) can be comprised between 2 and 88°, independently of one from the other. FIG. 3 (c) plots the ratio (ds/h=tan α1+tan α2) as a function of the axis angle α2 (with α2=30° and d0=0). It can be seen from FIG. 3 (c), that large axis angles (α1, α2) have the effect of increasing the ratio ds/h, which enhances the sensitivity of the measurement of ds for the determination of h, which is good for the accuracy of the determination of the implantation depth (h). The dashed line identifies ds=h. It is preferred that the axis angles (α1, α2) be selected such that the ratio ds/h≥1.

The light beams must travel through tissues before reaching the skin and forming the light projection patterns (51s, 52s). As the path length through tissues the light beams must cross increases, the light projection patterns (51s, 52s). become weaker and more blurred, because of light absorption and scattering through non-homogenous tissues. Large axis angles increase the length of the path the beam lights must travel through the tissues before reaching the skin of the patient which therefore reduces the sharpness of the light projection patterns (51s, 52s). To maintain an agreeable sharpness of the maxima (51m, 52m), it is therefore preferred that each axis angle (α1, α2) be not more than 60°, preferably not more than 45°. The shortest path from the main surface (20m) to the skin is defined by an axis angle, αi=0°, as shown in FIG. 5.

### Special Case #1: α1=α2

As illustrated in FIGS. 4 and 6, in case the at least first and second emission axes (X1, X2) form a same axis angle (i.e., α1=α2) with a normal axis (ni), Equations (1) and (1a) simplify to Equations (3) and (3a) as follows,

$$h = \frac{ds}{2 tan α1} - h 0 \qquad ( 3 )$$

$$h = \frac{ds - d 0}{2 tan α1} \qquad ( 3 a )$$

This configuration (i.e., α1=α2) is particularly interesting in case more than two light sources distributed at equal distances from a central point and forming a polygon projecting homothetically the light projection pattern (51s, 52s) onto the skin. It is also interesting for an infinite number of beams extending along a corresponding infinite number of emission axes (X1, X2) homothetically forming a circular light projection pattern (51s, 52s) as shown in FIG. 6. In these two embodiments, having first and second axis angles having a same value (i.e., α1=α2) renders the implantation depth measurement independent of the angle of rotation over the central normal axis (n0) of the external optical depth measuring device (40) relative to the AIMD.

In the embodiment illustrated in FIG. 6 a single light source projects a closed ring onto the skin (3s). With h0=0 and d0=0 (e.g., single light source), Equations (3) and (3a) simplify to Equation (3b),

$$h = \frac{ds}{2 tan α1} \qquad ( 3 b )$$

### Special Case #2: α1=0°

As shown in FIG. 5, the first axis angle can be nil (i.e., α1=) 0°. In other words, the first emission axis (X1) is perpendicular to the reference plane (R). It is mandatory that the second axis angle, α2+0°. Since tan 0=0, Equations (1) and (1a) simplify to Equations (4) and (4a) as follows,

$$h = \frac{ds}{tan α2} - h 0 \qquad ( 4 )$$

$$h = \frac{ds - d 0}{tan α2} \qquad ( 4 a )$$

This configuration (i.e., α1=) 0° is preferred for various reasons. First, the first light source (51) having an axis angle, α1=0°, yields the shortest path to the skin of the patient and can therefore advantageously be used also for communicating with an external controller. Second, if the external charger (10) is as described in WO2017202455 provided with an optical centring system discussed more in detail in continuation, requiring the AIMD to emit a light along an emission axis forming an axis angle α1=0°, the same first light source (51) can be used for first determining the implantation depth (h) of the AIMD, and then aligning the external charger such that the primary coil (11) and secondary coil (21) be centred coaxially (i.e., Z1=Z2).

In case a single light source is used, h0=d0=0 and Equations (4) and (4a) simplify to Equation (4b),

$$h = \frac{ds}{tan α2} \qquad ( 4 b )$$

### External Charger (10)

The determination of the implantation depth (h) of the AIMD is advantageous for tuning the external charger (10) so as to optimize resonance between the primary and secondary coils (11, 21) of the external charger and the AIMD, respectively. The external electric charger (10) is connectable to a source of electric current for feeding an alternative electric current to a primary coil (11) made of one or more wires coiled about a primary axis (Z1) and forming an external emitter (12) of magnetic waves.

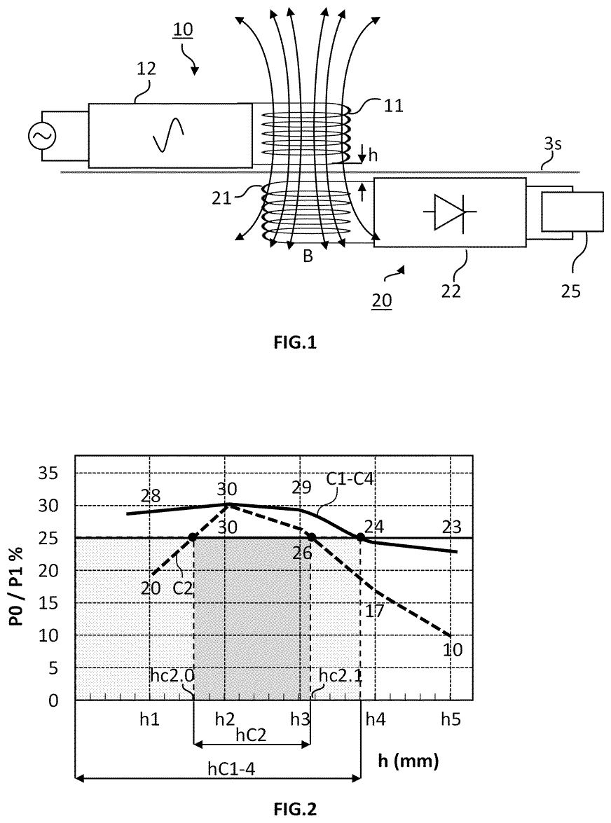

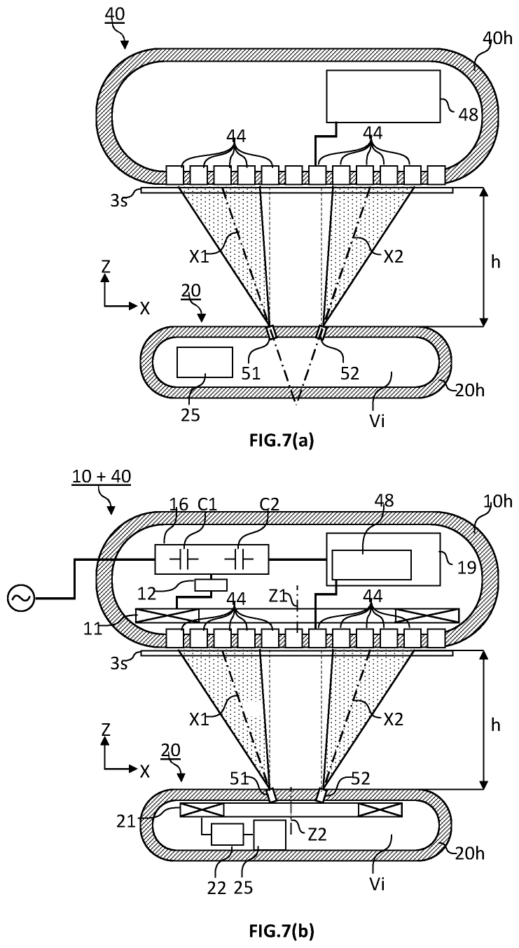

As described supra and illustrated in FIGS. 5, 6, 7 (a), and 7 (b), the AIMD (20) comprises a secondary coil (21) made of one or more wires coiled about a secondary axis (Z2) normal to the reference plane (R) and forming an implanted receiver (22). As shown in FIGS. 1 and 7 (b), when the electric charger (10) is located adjacent to the AIMD with the primary axis (Z1) positioned coaxially with the secondary axis (Z2) and when the primary coil is fed with electric current, a magnetic link is formed and an electric current is induced in the secondary coil, which serves to power the AIMD or to recharge a rechargeable battery (25) enclosed in the inner volume (Vi).

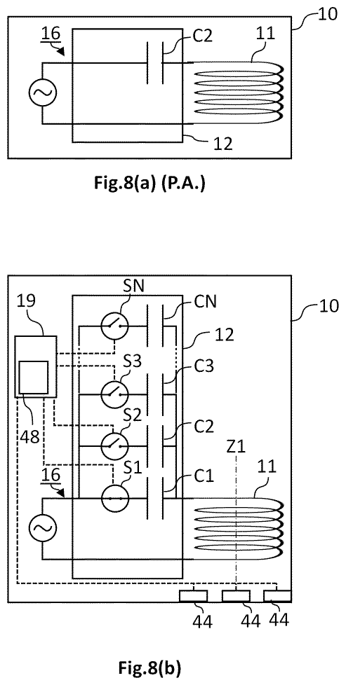

The external charger can comprise a charger housing (10h) enclosing an electrical circuit (16) configured for varying the properties of the electric current fed to the primary coil (11), as shown in FIGS. 7 (b) and 8 (b). A processor (19) of the external charger (10) is configured for retrieving the implantation depth (h) of the AIMD (20) measured by the external optical depth measuring device (40) and for controlling the current fed to the primary coil (11) to optimize a power transfer efficiency (P0/P1) between the primary coil (11) of the external charger (10) and the secondary coil (21) of the AIMD as a function of the implantation depth (h).

The power transfer efficiency and the charging current (which affects the charging time) depends inter alia on the implantation depth (h). Indeed, the electrical circuit connected to each of the primary and secondary coils (11, 21) contain charging capacitors which capacitance values should be precisely tuned to enhance the resonance between the external charger (10) and the AIMD (20). Resonance occurs when the natural frequency of the system, which depends inter alia on the implantation depth (h), is the same as the frequency of the electromagnetic force applied to the system by the oscillating magnetic field (B). Achieving resonance allows maximizing the amplitude of the oscillating response of the system. FIG. 8 (a) shows a simplified electrical circuit (16) of an external charger of the prior art comprising a single charging capacitor (C2) (the electrical circuit comprises other capacitors performing other functions, but in the present simplified representation of FIG. 8 (a), only charging capacitor (C2) is shown). Referring to FIG. 2, the dashed curve labelled “C2” shows the power transfer efficiency (P0/P1) as a function of the implantation depth (h) for a system comprising a prior art external charger according to FIG. 8 (a), with a single charging capacitor (C2) of fixed capacitance. Setting (arbitrarily) a limit of acceptable P0/P1 value of P0/P1=25%, it can be seen in FIG. 2 that the external charger (10) is optimized for a limited C2-implantation depth range (hC2), extending between a lowest boundary depth value (hc2.0) and a highest boundary value (hc2.1). Since external chargers (10) are produced industrially and not tailored to a specific application, such one-fit-them-all external chargers will be suboptimal for all AIMD's implanted at an implantation depth falling out of the C2-implantation depth (hC2). There is no way to change the capacitance of charging capacitor (C2) of the external charger to optimize the power transfer efficiency (P0/P1). In such conditions, determining the implantation depth (h) of the AIMD may be interesting from an academic point of view or for a number of other reasons, but not for optimizing the power transfer efficiency (P0/P1) between the primary coil (11) of the external charger (10) and the secondary coil (21) of the implanted AIMD (20).

External Charger with Several Charging Capacitors (C1-CN)

By contrast, the electrical circuit (16) of a preferred external charger (10) of the present invention illustrated in FIG. 8 (b) comprises at least first and second charging capacitors (C1-CN) of same or different capacitances, which can be individually connected or disconnected to the electrical circuit (16). Typically, the charging capacitors are arranged in parallel with switches (S1-SN) which can be closed or opened to connect or disconnect each corresponding charging capacitor (C1-CN) to the electrical circuit (16). The processor (19) of the external charger (10) is configured for optimizing the power transfer efficiency between the primary coil (11) of the external charger (10) and the secondary coil (21) of the AIMD (20) as a function of the implantation depth (h) by selecting which of charging capacitors (C1, C2) to connect to the electrical circuit (16). Once the best charging capacitors combination is selected, the processor is configured for optimizing the power supplied to the transmitter circuit to yield a nominal charging current of the AIMD.

In a preferred embodiment, the electrical circuit (16) comprises N charging capacitors (C1, C2 . . . . CN) wherein N is preferably comprised between 3 and 8, more preferably between 4 and 6. The N charging capacitors can have same or different capacitances. They can be individually connected to or disconnected from the electrical circuit or a selection of charging capacitors forming a group can be connected to or disconnected from the electrical circuit. The processor (19) is configured for determining which combination of one or several of the N charging capacitors (C1, C2 . . . . CN) to connect to the electrical circuit (16), such as to optimize the power transfer efficiency as a function of the implantation depth (h), The processor (19) can control the status of the switches (S1-SN) to implement the thus determined combination.

Referring to FIG. 2, the solid curve labelled “C1-C4” shows the power transfer efficiency (P0/P1) as a function of the implantation depth (h) for a system comprising an external charger according to the present invention and to FIG. 8 (b), with four charging capacitors (C1-C4) of fixed capacitance arranged in parallel with corresponding switches (S1-S4) The combinations of charging capacitors (C1-C4) connected at one time to the electrical circuit (16) was changed for different implantation depths (h) and selected so as to yield the capacitance optimizing the resonance between the primary and secondary coils (11, 21). Setting the limit of acceptable P0/P1 value of P0/P1=25%, as for the dashed curve discussed supra with respect to FIG. 8 (a) of the prior art, it can be seen in FIG. 2 that the external charger (10) is optimized over a much broader C1-4-implantation depth range (hC1-4), extending over both lowest and highest boundary depth values (hc2.0, hc2.1) of the prior art C2-implantation depth range (hC2) discussed supra with respect to the dashed curve of FIG. 2 corresponding to the prior art electrical circuit of FIG. 8 (a),

It can be seen in FIG. 2 that the implantation depth range is more than doubled with the external charger (10) having an electrical circuit (16) according to the present invention as illustrated in FIG. 8 (b), compared with the prior art external charger of FIG. 8 (a) (i.e., hC1-4>2 hC2). This represents a breakthrough as the charging operation of the implanted battery of an AIMD can be performed with a same efficacy regardless of the physiognomy of the patient (lean, fat, muscular, thick/thin featured, etc.) or of the surgeon who implanted the AIMD, which can affect the implantation depth (h) of the AIMD.

External Charger with Optical Centering System

In a preferred embodiment, illustrated in FIG. 7 (b), the external optical depth measuring device (40) is included in the external charger (10), and the intelligence (48) is included in the processor (19). The external charger therefore also comprises the two or more photodetectors (44) or the array of two or more photodetectors of the external optical depth measuring device (40) which are configured for detecting the at least first and second maxima (51m, 52m) of the light projection pattern emitted by the one or more light sources.

In a preferred embodiment, the external charger comprises at least three photodetectors (44) and comprises a module for centring the external charger (10) with respect to the implanted AIMD (20) as described in WO2017202455. The at least three photodetectors (44) form a polygon of N edges, normal to the primary axis (Z1), and which centroid belongs to the primary axis (Z1). The module comprises an indicator indicating how the external charger is to be displaced over the surface of the skin of a patient to position the external charger with the primary axis, (Z1), being coaxial with the secondary axis (Z2), of the AIMD, as a function of the energy received by each of the at least three photodetectors from the light beam emitted by a central light source of the AIMD.

The position of the secondary axis (Z2) centred on the secondary coil (21) of the AIMD can be univocally determined from the position of a central light source. Preferably, the central light source belongs to the secondary axis (Z2) or is offset therefrom by a known distance and angle. The central light source emits a light beam perpendicular to the reference plane (R), i.e., the axis angle of the central light beam must be 0°. The central light source can be different from the one or more light sources (51, 52) used for determining the implantation depth (h). It is, however, preferred that the central light source be one of the one or more light sources (51, 52) which must have an axis angle, α1=0°. For example, the central light source could be the first light source (51) of FIG. 5.

With this embodiment, the external charger (10) including the external optical depth measuring device (40) can be used for continuously and sequentially
aligning the external charger with the AIMD (20),
determining the implantation depth (h) of the AIMD,
optimizing the electrical circuit (16) to approach and preferably reach resonance and
finally, recharging the implanted battery as efficiently and comfortably as possible to date. A rapid battery charging operation is beneficial to the comfort of the patients.

### Method for Determining an Implantation Depth (h) of an Aimd

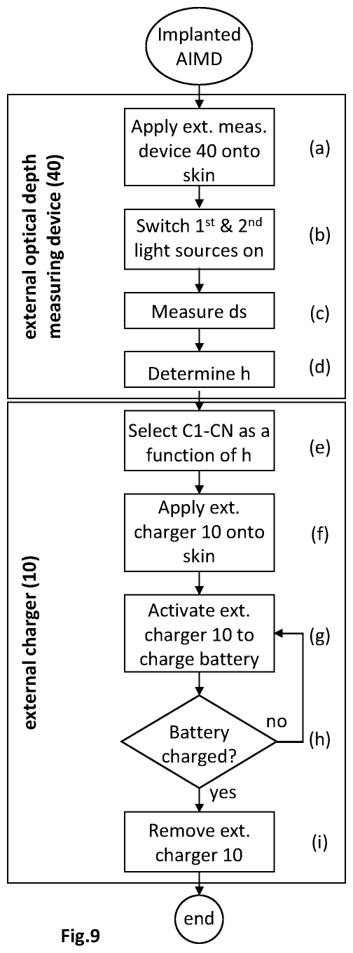

The present invention also concerns a method for determining an implantation depth (h) separating an external optical measuring device (40) as described supra from an AIMD implanted in a patient. The method is illustrated in the flowchart of FIG. 9 and requires that the implanted AIMD be as discussed supra, comprising one or more light sources (51, 52) as defined supra. As shown in FIG. 9 (a), the external optical depth measuring device (40) is applied onto the skin of the patient, vis-à-vis the AIMD. The controller activates the one or more light sources (51, 52) of the AIMD (cf. FIG. 9 (b)) and measures the maxima distance (ds) separating the at least first and second maxima (51m, 52m) (cf. FIG. 9 (c)). As shown in FIG. 9 (d), the value of the implantation depth can then be determined as explained with reference to FIGS. 3 to 5.

Once the implantation depth (h) has been determined, the power transfer efficiency between a primary coil (11) of an external charger (10) and a secondary coil (55) of the AIMD (20) can be optimized as a function of the thus determined implantation depth (h), as follows. An external charger (10) as defined above is provided. Based on the implantation depth (h) thus determined, an optimal electric current is determined to be fed to the primary coil (11) as a function of the implantation depth (h) (cf. FIG. 9 (e)), to optimize the power transfer efficiency (P0/P1) between the primary coil (11) of the external charger (10) and the secondary coil (21) of the AIMD. As indicated in FIG. 9 (f), the external charger (10) can be laid onto the skin vis-à-vis the AIMD and the optimal electric current can be fed to the primary coil (11) to induce a current in the secondary coil (21) (cf. FIG. 9 (g)). The feeding of electric current is carried out until a desired level of charging of the battery is reached (cf. loop (g)-(h)), at which point the external charger (10) can be removed, as indicated in FIG. 9 (i)).

The optimal current fed to the primary coil (11) can be controlled by selecting one or more charging capacitors (C1-CN) to connect to the electrical circuit (16), such as to optimize the power transfer efficiency between the primary coil (11) of the external charger (10) and the secondary coil (21) of the AIMD (20) as a function of the distance thus determined. The controller controls the switches (S1-SN) to connect or disconnect the corresponding charging capacitors to the electrical circuit.

| REF. # | DESCRIPTION |
|---|---|
| 3s | Skin |
| 10 | External charger |
| 11 | Primary coil |
| 12 | Transmitter |
| 16 | Electrical circuit of the external charger |
| 19 | Processor (external charger) |
| 20 | AIMD |
| 20e | Encapsulation unit |
| 20h | Housing of AIMD |
| 20m | Main surface of housing |
| 21 | Secondary coil |
| 22 | Receiver |
| 25 | Implanted rechargeable battery |
| 40 | External optical depth measuring device |
| 40h | Housing of external charger |
| 44 | Photodetector of the external optical depth measuring device |
| 48 | Intelligence of the external optical depth measuring device |
| 51, 52 | First and second light sources |
| 51m, 52m | First and second maxima |
| 51s, 52s | First and second light projection patterns |
| C1-CN | First to N^{th} charging capacitors |
| d0 | Distance separating first and second light sources |
| D1, D2 | Distance separating the first and second light sources from |
| | central normal axis n0 |
| ds | Maxima distance |
| h | Implantation depth and distance between primary and |
| | secondary coils |
| hC1-4 | C1-4-implantation depth range |
| hC2 | C2-implantation depth range |
| hc2.0 | hC2 lower boundary |
| hc2.1 | hC2 upper boundary |
| n0 | Central normal axis |
| n1, n2 | Normal axes intercepting first and second light sources, |
| | respectively |
| S1-SN | First to N^{th} switches |
| Vi | Inner volume |
| X1, X2 | First and second emission axes |
| X, Y, Z | Space reference axes |
| Z1-Z2 | Primary and secondary axes of the corresponding coils |
| α1, a2 | Axis angles |

1. System for optically measuring an implantation depth (d) of an active implantable medical device (AIMD) below a skin (3s) of a patient implanted with the AIMD, wherein the AIMD (20) comprises,
an encapsulation unit (20e) suitable for being subcutaneously implanted with a reference plane (R) of the encapsulation unit being substantially parallel to the skin (3s) of the patient when implanted in the patient and comprising a housing (20h) comprising walls defining an inner volume (Vi) sealed from an outer environment by the walls and defining a first main surface (20m) configured for facing the skin of the patient when implanted in the patient, characterized in that, the AIMD comprises,
one or more light sources (51, 52) configured for emitting one or more light beams from the main surface (20m) towards the outer environment to form a light projection pattern (51s, 52s) at a level of the skin (3s) of the patient, characterized by an intensity profile comprising at least first and second maxima (51m, 52m) separated from one another by a maxima distance (ds), wherein
the one or more light sources are configured for emitting one or more light beams along at least two emission axes (X1, X2) defined as the axes extending from one or more emission points (51, 52) of the one or more light sources to the corresponding at least first and second maxima (51m, 52m) of the light projection pattern (51s, 52s) at the level of the skin (3s) of the patient, wherein the at least two emission axes (X1, X2) are non-parallel to one another, and form at least first and second axis angles (α1, α2) relative to a normal axis (n1; n2) perpendicular to the reference plane (R), each comprised between 0° and 88°, preferably between 10° and 60°, more preferably between 20° and 45°, and in that, the system comprises an external optical depth measuring device (40) comprising
a controller configured for activating the one or more light sources of the AIMD, and
two or more photodetectors (44) or an array of two or more photodetectors configured for detecting the at least first and second maxima (51m, 52m) of the light projection pattern emitted by the one or more light sources, and
an intelligence (48) configured for,
determining the respective positions of the first and second maxima (51m, 52m), o measuring the maxima distance (ds) between the first and second maxima (51m, 52m) at the level of the skin (3s) of the patient, and
determining from the maxima distance (ds) the implantation depth (h) of the AIMD below the skin (3s) of the patient.

2. System according to claim 1, wherein the implantation depth (h) is determined with any one of the following expressions (1) or (1a),
$$h = \frac{ds}{tan α1 + tan α2} - h 0 , or \qquad ( 1 )$$
$$h = \frac{ds - d 0}{tan α1 + tan α2} \qquad ( 1 a )$$
wherein
h0 is a distance measured along a central normal axis (n0) perpendicular to the reference plane (R) between the intersection point of the first and second emission axes (X1, X2) and the main surface (20m), and
d0 is a distance separating the first from the second light sources (51, 52).

3. System according to claim 2, wherein the first and second axis angles (α1,α2) are equal (i.e., (α1=α2), and wherein the implantation depth (h) is determined with any one of the following expressions (3) or (3a),
$$h = \frac{ds}{2 tan α1} - h 0 \qquad ( 3 )$$
$$h = \frac{ds - d 0}{2 tan α1} . \qquad ( 3 a )$$

4. System according to claim 2, wherein the first axis angle (α1) is equal to and is different from the second axis angle (@2) and wherein the implantation depth (h) is determined with any one of the following expressions (4) or (4a),
$$h = \frac{ds}{tan α2} - h 0 \qquad ( 4 )$$
$$h = \frac{ds - d 0}{tan α2} . \qquad ( 4 )$$

5. System according to claim 1, comprising either, at least first and second light sources (51, 52) separated from one another by a distance (d0) and each configured for emitting a light beam along the corresponding at least first and second emission axes (X1, X2), or a single light source (51) a single light source (51) configured for emitting a light beam characterized by the at least first and second maxima (51m, 52m).

6. System according to claim 1,
comprising an external charger (10) comprising or connectable to a source of power for feeding alternative current to a primary coil (11) made of one or more wires coiled about a primary axis (Z1) and forming an external emitter (12) of magnetic waves,
wherein the AIMD (20) comprises a secondary coil (21) made of one or more wires coiled about a secondary axis (Z2) normal to the reference plane (R) and forming an internal receiver (22), such that when the electric charger is located adjacent to the AIMD with the primary axis (Z1) positioned coaxially to the secondary axis (Z2) and when the primary coil is fed with alternative current, a magnetic link is formed and an alternative current induced in the secondary coil is optimized, which serves to power the AIMD or to recharge a rechargeable battery (25) enclosed in the inner volume (Vi).

7. System according to the preceding claim 6, wherein the external charger comprises,
an electrical circuit (16) configured for varying the properties of the alternative current fed to the primary coil (11),
a processor (19) configured for retrieving the implantation depth (h) of the AIMD (20) measured by the external optical depth measuring device (40) and for controlling the alternative current fed to the primary coil (11) to optimize a power transfer efficiency between the primary coil (11) of the external charger (10) and the secondary coil (21) of the AIMD as a function of the implantation depth (h).

8. System according to the preceding claim 7 wherein,
the electrical circuit (16) comprises at least first and second charging capacitors (C1, C2) of given capacitances, which can be individually connected or disconnected to the electrical circuit,
the processor (19) is configured for optimizing the power transfer efficiency between the primary coil (11) of the external charger (10) and the secondary coil (21) of the AIMD (20) as a function of the implantation depth (h) by selecting which of the first and I or second capacitance (C1, C2) to connect to the electrical circuit (16).

9. System according to the preceding claim 8, wherein the electrical circuit (16) comprises N charging capacitors (C1, C2 . . . . CN) of same or different charging capacitors, which can be connected or disconnected individually or in groups of charging capacitors to the electrical circuit, and wherein the processor (19) is configured for determining which combination of one or several of the N charging capacitors (C1, C2 . . . . CN) to connect to the electrical circuit (16), such as to optimize the power transfer efficiency as a function of the implantation depth (h), wherein N is preferably comprised between 3 and 8, more preferably between 4 and 6.

10. System according to claim 6, wherein the external optical depth measuring device (40) is included in the external charger (10), and wherein the intelligence (48) is included in the processor (19).

11. Method for determining an implantation depth (h) separating an external optical measuring device (40) from an active implantable medical device (=AIMD) implanted in a patient, comprising the following steps,
providing an already implanted AIMD (20) as defined in a system according to claim 1, whose main surface (20m) is separated from an outer environment by a distance to be determined, by a membrane substantially parallel to the reference plane (R),
applying an external depth measuring device (40) as defined in the system according to claim 1 onto the membrane, vis-a-vis the AIMD,
activating with the controller the one or more light sources (51, 52) of the AIMD,
measuring the maxima distance (ds) and determining therefrom the value of the distance.

12. Method according to the preceding claim 11, further comprising the following steps for optimizing a power transfer efficiency between a primary coil (11) of an external charger (10) and a secondary coil (55) of the AIMD (20) as a function of the implantation depth (h),
providing an external charger (10) as defined in the system according to claim 10, retrieving the thus determined distance, and
determining an optimal alternative current to be fed to the primary coil (11) as a function of the distance, to optimize the power transfer efficiency (P0/P1) between the primary coil (11) of the external charger (10) and the secondary coil (21) of the AIMD,
applying the external charger (10) onto the membrane vis-a-vis the AIMD and feeding the optimal alternative current to the primary coil (11).

13. Method according to the preceding claim 12, wherein the system is according to claim 8, and wherein controlling the alternative current fed to the primary coil (11) comprises selecting one or more charging capacitors (C1-CN) to connect to the electrical circuit (16), such as to optimize the power transfer efficiency between the primary coil (11) of the external charger (10) and the secondary coil (21) of the AIMD (20) as a function of the distance thus determined.
